(19) United States
(12) Patent Application Publication
(10) Pub. No.: US 2025/0196105 A1
(43) Pub. Date: Jun. 19, 2025

(54) CHROMIUM-ON-ALUMINA CATALYSTS FOR HYDROCARBON DEHYDROGENATION

(71) Applicant: CLARIANT INTERNATIONAL LTD, Muttenz (CH)

(72) Inventors: Rong Xing, Louisville, KY (US); Vladimir Fridman, Louisville, KY (US); Matthew J. Greaney, Hayward, CA (US); Kuirun Zhang, Hayward, CA (US); Aaron B. Miller, San Ramon, CA (US); Claus G. Lugmair, Santa Cruz, CA (US)

(21) Appl. No.: 18/923,741

(22) Filed: Oct. 23, 2024

Related U.S. Application Data
(60) Provisional application No. 63/609,462, filed on Dec. 13, 2023.

(51) Int. Cl. B01J 23/26; B01J 23/00; B01J 23/06; B01J 37/02; B01J 37/10; C07C 5/32

(52) U.S. Cl. CPC B01J 23/26; B01J 23/002; B01J 23/06; B01J 37/0236; B01J 37/024; B01J 37/10; C07C 5/324; B01J 2523/12; B01J 2523/43; B01J 2523/48; B01J 2523/67; C07C 2523/86

(57) ABSTRACT

The present disclosure provides a dehydrogenation catalyst composition and methods for making and using it. One aspect provides a dehydrogenation catalyst composition comprising a zinc-doped alumina carrier; and associated with the carrier, chromium, tin, zirconium and alkali metal, wherein the catalyst composition comprises: aluminum in an amount in the range of 51-88 wt %, calculated on an Al₂O₃ basis; zinc in an amount in the range of 0.1-10 wt %, calculated on a ZnO basis; chromium in an amount of 12-30 wt %, calculated on a Cr₂O₃ basis; tin in an amount in the range of 0.005-2 wt %, calculated on a SnO₂ basis; zirconium in an amount in the range of 0.1-2 wt %, calculated on a ZrO₂ basis; and alkali metal in an amount in the range of 0.1-5 wt %, calculated on a M₂O basis.

### CROSS-REFERENCE TO RELATED APPLICATIONS

This application claims the benefit of priority of U.S. Provisional Patent Application No. 63/609,462 filed Dec. 13, 2023, which is hereby incorporated herein by reference in its entirety.

### BACKGROUND OF THE DISCLOSURE

### Field of the Disclosure

The present disclosure relates generally to hydrocarbon dehydrogenation catalyst compositions. More particularly, the present disclosure relates to chromium-on-alumina catalyst compositions containing, among other possible components, zinc and tin, to methods of making such catalyst compositions, and to methods for dehydrogenating alkanes with such catalysts.

### Technical Background

Alkane dehydrogenation, for example, using the so-called “Houdry process,” is a chemically useful process for the production of various unsaturated hydrocarbon products. For example, propane, isobutane, and isopentane can be catalytically dehydrogenated to propylene, isobutylene, and amylene. There are a variety of uses for such products; for example, propylene can later be used for the production of polymers, isobutylene can be used to make jet fuel, and amylene can be further reacted to produce pharmaceutical solvents. However, dehydrogenation of light alkanes is highly endothermic, often requiring temperatures in excess of 500° C. to be economically feasible, with heat continually supplied to sustain the reaction. Additionally, the rate of reaction is usually slow even at high temperatures. To address this problem, a number of different types of catalytic processes have been developed to speed up alkane dehydrogenation. Two common types involve chromium-on-alumina catalysts and supported precious metal catalysts, e.g. Pt/Al₂O₃.

Alumina is frequently used as a catalyst carrier. Chromium-on-alumina dehydrogenation catalysts in particular have been in use for several decades. In addition to chromium and aluminum, a number of other components are frequently added to these dehydrogenation catalysts. Because catalytic dehydrogenation is performed at extremely high temperatures, thermally stable materials are desirable. There are a number of known catalysts in which a variety of components, e.g., zirconia, silica, and alkali metal oxides, are added to improve the thermal stability of the catalyst material.

Despite the commercial utility of efficient catalytic dehydrogenation processes, existing catalyst materials often present tradeoffs. Thus there is an ongoing need in the field for dehydrogenation catalysts that exhibit desirable selectivity and yield toward light alkanes.

### SUMMARY OF THE DISCLOSURE

The scope of the present disclosure is not affected to any degree by the statements within the summary.

In one aspect, the disclosure provides a dehydrogenation catalyst composition comprising a zinc-doped alumina carrier, and, associated with the carrier, chromium, tin, zirconium, and alkali metal. In various embodiments, the catalyst composition comprises:
aluminum in an amount in the range of 51-88 wt %, calculated on an Al₂O₃ basis,
zinc in an amount in the range of 0.1-10 wt % calculated on a ZnO basis,
chromium in an amount of 12-30 wt % calculated on a Cr₂O₃ basis,
tin in an amount in the range of 0.005-2 wt % calculated on a SnO₂ basis,
zirconium in an amount in the range of 0.1-2 wt % calculated on a ZrO₂ basis, and
alkali metal in an amount in the range of 0.1-5 wt % calculated on a M₂O basis.
   Some embodiments also include silicon in an amount in the range of 0.1-5 wt % calculated on a SiO₂ basis, and/or alkaline earth metal in an amount in the range of 0.1-5 wt % calculated on a MO basis.

In another aspect, the disclosure provides a method for preparing a dehydrogenation catalyst as described herein, the method comprising: preparing a calcined zinc-doped alumina carrier; then impregnating the zinc-doped alumina carrier with chromium, zirconium, tin and alkali metal; then calcining the impregnated carrier.

Another aspect of the disclosure provides a process for catalytically dehydrogenating hydrocarbons, the process comprising contacting a gaseous aliphatic hydrocarbon stream comprising at least one C₂-C₅ alkane with the catalyst under conditions sufficient to dehydrogenate at least a portion of the C₂-C₅ alkane to one or more corresponding C₂-C₅ alkenes, e.g. at a temperature from 520° C. to 680° C., a pressure from 0.2 bar to 2.0 bar, and an LHSV from 0.8 h⁻¹ to 2.5 h⁻¹. The catalyst compositions provided, prepared and used according to the methods provided, exhibit greater selectivity than prior art catalysts.

Other aspects of the disclosure will be apparent to the person of ordinary skill in the art in view of the disclosure herein.

### BRIEF DESCRIPTION OF FIGURES

FIG. 1 presents three XPS spectra of a Zn-doped alumina catalyst, ZnAl₂O₄, and ZnO, from top to bottom, respectively.

### DETAILED DESCRIPTION

The present inventors have noted that, while many chromium-on-alumina catalysts were known, one common challenge is selectivity for dehydrogenation with respect to other products. For example, typical chromium-on-alumina catalysts often have limitations in propylene selectivity in the dehydrogenation of propane. These limitations in propylene selectivity lead to relatively high downstream separation cost. Thus, a high-selectivity dehydrogenation catalyst is very desirable for commercial processes such as paraffin dehydrogenation processes.

The present inventors have found that use of a zinc-doped alumina support together with the use of tin in the catalyst can provide dehydrogenation catalysts exhibiting improved alkene selectivity, for example, greater propylene selectivity than commercially available catalysts for propane dehydrogenation. Such catalyst may thus be a desirable alternative to existing catalyst compositions for dehydrogenating light alkanes.

### I. Composition

Accordingly, one aspect of the disclosure provides a dehydrogenation catalyst composition comprising a zinc-doped alumina carrier; and, associated with the carrier, chromium, tin, zirconium, alkali earth metal, and alkali metal.

One particular aspect of the disclosure provides a dehydrogenation catalyst composition comprising a zinc-doped alumina carrier; associated with the carrier, chromium, tin, zirconium and alkali metal, wherein the catalyst composition comprises: aluminum in an amount in the range of 51-88 wt %, calculated on an Al₂O₃ basis; zinc in an amount in the range of 0.1-10 wt % calculated on a ZnO basis; chromium in an amount of 12-30 wt % calculated on a Cr₂O₃ basis; tin in an amount in the range of 0.005-2 wt % calculated on a SnO₂ basis; zirconium in an amount in the range of 0.1-2 wt % calculated on a ZrO₂ basis; alkali metal in an amount in the range of 0.1-5 wt % calculated on a M₂O basis; alkali earth metal in an amount in the range of 0.1-2 wt % calculated on a MO basis.

Unless otherwise indicated, regardless of the actual stoichiometry of the oxide, oxides are calculated as the most stable oxide for purposes of weight percent determinations. For example, the person of ordinary skill in the art will appreciate that a non-stoichiometric oxide of aluminum, or even another form of aluminum, may still be calculated as Al₂O₃ for purposes of weight percent determinations. Moreover, unless otherwise indicated, the compositions are described on an as-calcined basis.

The catalyst composition of this aspect of the disclosure includes a zinc-doped alumina carrier. Alumina is useful as a carrier for dehydrogenation catalysts because of its thermal stability, among other desirable properties. The crystal structure of alumina may vary according to how it is prepared and treated. Some alumina structures exhibit greater or lesser catalytic activity. Accordingly, in various embodiments, alumina is present as one or more of gamma-alumina, alpha-alumina, theta-alumina, delta-alumina, eta-alumina, kappa-alumina, and chi-alumina, and/or in the form of an aluminate, e.g. as zinc aluminate, as described in more detail below. For example, in various embodiments, alumina is present as gamma-alumina, theta-alumina and/or eta-alumina.

In various aspects of compositions of the disclosure, the zinc-doped alumina is present in a substantial portion of the catalyst, e.g., in an amount in the range of 51-88 wt %, calculated on an Al₂O₃ basis. The amount of aluminum can vary, for example, based on the amounts of other components provided in the catalyst. In various embodiments as otherwise described herein, aluminum is present in the catalyst in an amount in the range of 51-85 wt %, or 51-82 wt %, or 51-79 wt %, or 60-88 wt %, or 60-85 wt %, or 60-82 wt %, or 60-79 wt %, or 68-88 wt %, or 68-85 wt %, or 68-82 wt %, or 68-79 wt %, or 73-88 wt %, or 73-85 wt %, or 73-82 wt %, or 73-79 wt %, calculated on an Al₂O₃ basis. For example, in some embodiments aluminum is present in an amount in the range of 68-85 wt %, calculated on an Al₂O₃ basis.

The present inventors unexpectedly found that inclusion of zinc in the alumina carrier helped to enhance catalyst dehydrogenation selectivity. Accordingly, in one aspect of the disclosure, the catalyst composition includes in the range of 0.1-10 wt % zinc, calculated on a ZnO basis. The amount of zinc can vary, and the person of ordinary skill in the art can determine an appropriate amount based on the present disclosure. In various embodiments as otherwise described herein, zinc is present in the catalyst in an amount in the range of 0.1-8 wt %, or 0.1-6 wt %, or 0.1-4 wt %. In various embodiments as otherwise described herein, zinc is present in the catalyst in an amount in the range of 0.5-10 wt %, e.g., 0.5-8 wt %, or 0.5-6 wt %, or 0.5-4 wt %, calculated on a ZnO basis. In various embodiments as otherwise described herein, zinc is present in the catalyst in an amount in the range of 1-10 wt %, e.g., 1-8 wt %, or 1-6 wt %, or 1-4 wt %, calculated on a ZnO basis. In various embodiments as otherwise described herein, zinc is present in the catalyst in an amount in the range of 2-10 wt %, e.g., 2-8 wt %, or 2-6 wt %, or 2-4 wt %, calculated on a ZnO basis. In some specific embodiments, zinc is present in the catalyst in an amount of 0.5-6 wt % (e.g., 1-6 wt %), calculated on a ZnO basis.

In various embodiments, zinc is present in the catalyst predominantly in the form of ZnAl₂O₄ as measured by X-ray photoelectron spectroscopy (XPS). For example, In various embodiments, at least 75 mol % of the zinc is present in the catalyst in the form of ZnAl₂O₄, e.g., at least 85 mol % or at least 95 mol %, as measured by XPS. As the person of ordinary skill in the art will appreciate, x-ray photoelectron spectroscopy (XPS) can be used to probe the chemical state of a given species in a compound or mixture, especially at the surface of a material. The person of ordinary skill in the art can use conventional techniques, to determine the amount of zinc present as ZnAl₂O₄, e.g., by fitting of known XPS spectra for ZnAl₂O₄ and zinc oxides to an experimental trace. As described in more detail below, the present inventors have noted that various catalysts of the Examples have a high proportion of zinc present as ZnAl₂O₄ as measured by XPS.

Chromium is generally understood to be an active catalyst species in dehydrogenation processes. In various aspects of the disclosure, chromium is present in the catalyst composition in an amount in the range of 12-30 wt %, calculated on a Cr₂O₃ basis. The amount can be varied by the person of ordinary skill in the art. For example, in various embodiments as otherwise described herein, chromium is present in the catalyst in an amount in the range of 12-28 wt %, or 12-25 wt %, or 12-22 wt %, calculated on a Cr₂O₃ basis. In various embodiments as otherwise described herein, chromium is present in the catalyst in an amount in the range of 15-30 wt %, e.g., 15-28 wt %, or 15-25 wt %, or 15-22 wt %, calculated on a Cr₂O₃ basis. In various embodiments as otherwise described herein, chromium is present in the catalyst in an amount in the range of 17-30 wt %, e.g., 17-28 wt %, or 17-25 wt %, or 17-22 wt %, calculated on a Cr₂O₃ basis. In various particular embodiments, chromium is present in the catalyst in an amount in the range of 15-25 wt %, calculated on a Cr₂O₃ basis.

The present inventors also unexpectedly found that inclusion of tin in the catalyst helped to enhance catalyst dehydrogenation selectivity. Accordingly, in one aspect of the disclosure, the catalyst composition includes in the range of 0.005-2 wt % tin, calculated on a SnO₂ basis. The amount of tin can vary, and the person of ordinary skill in the art can determine an appropriate amount based on the present disclosure. In various embodiments as otherwise described herein, tin is present in the catalyst in an amount in the range of 0.005-1 wt %, e.g., 0.005-0.7 wt %, or 0.005-0.5 wt %, calculated on a SnO₂ basis. In various embodiments as otherwise described herein, tin is present in the catalyst in an amount in the range of 0.05-2 wt %, e.g., 0.05-1 wt %, or 0.05-0.7 wt %, or 0.05-0.5 wt %, calculated on a SnO₂ basis. In various embodiments as otherwise described herein, tin is present in the catalyst in an amount in the range of 0.1-2 wt %, or 0.1-1 wt %, or 0.1-0.7 wt %, calculated on a SnO₂ basis. In various embodiments as otherwise described herein, tin is present in the catalyst in an amount in the range of 0.2-2 wt %, e.g., or 0.2-1 wt %, or 0.2-7 wt %, calculated on a SnO₂ basis. In various specific embodiments, tin is present in an amount of 0.1-1 wt %, calculated on a SnO₂ basis.

Zirconium can improve the thermal stability of the catalysts, allowing them to be operated at higher temperatures and/or have a longer life. In various embodiments as otherwise described herein, zirconium is present in the catalyst in an amount in the range of 0.1-1.7 wt %, or 0.1-1.5 wt %, or 0.1-1.2 wt %, or 0.2-2 wt %, or 0.2-1.5 wt %, or 0.2-1.2 wt %, or 0.4-2 wt %, or 0.4-1.5 wt %, or 0.4-1.2 wt %, calculated on a ZrO₂ basis. In some specific embodiments, zirconium is present in the catalyst in an amount in the range of 0.2-1.2 wt % on a ZrO₂ basis, calculated on a ZrO₂ basis.

Alkali metal additives can also improve the thermal stability of the catalysts. In various embodiments as otherwise described herein, the alkali metal is present in the catalyst in an amount in the range of 0.1-3 wt %, or 0.1-2 wt %, or 0.1-1 wt %, or 0.2-5 wt %, or 0.2-3 wt %, or 0.2-2 wt %, or 0.2-1 wt %, or 0.4-5 wt %, or 0.2-3 wt %, or 0.2-2 wt %, or 0.2-1 wt %, calculated on an M₂O basis. In some specific embodiments, the alkali metal is present in the catalyst in an amount in the range of 0.2-1.5 wt %, calculated on an M₂O basis. In some of those embodiments, the alkali metal is present in the catalyst in an amount of 0.2-1 wt %, calculated on an M₂O basis. Sodium and potassium are frequent choices of alkali metal additives in the art. Thus in various embodiments the alkali metal is Na and/or K. In some specific embodiments, the alkali metal is Na. In other specific embodiments, the alkali metal is K.

The present inventors have noted that addition of silica to the carrier material may be desirable in some cases. Hence, in various embodiments as otherwise described herein, the carrier is a zinc-doped silica/alumina carrier, and the catalyst comprises silicon, present in an amount up to 5 wt %, calculated on an SiO₂ basis. In some specific embodiments, silicon is present in the catalyst in an amount of up to 3 wt %, e.g., or up to 1 wt %, calculated on an SiO₂ basis. In other embodiments silicon is present in the catalyst in an amount of 0.1-5 wt %, e.g., 0.1-3 wt %, or 0.1-1 wt %, or 0.2-5 wt %, or 0.2-3 wt %, or 0.2-1 wt %, calculated on an SiO₂ basis.

However, the present inventors have also noted that advantageous catalyst compositions can be made without substantial use of silicon. For example, in various embodiments as otherwise described herein, silicon is present in the catalyst in an amount no more than 0.5 wt % silicon, e.g. 0.2 wt %, or no more than 0.1 wt %, or no more than 0.05 wt %, calculated on an SiO₂ basis.

The present inventors note that alkaline earth metal additives can improve the thermal stability of the catalysts. Accordingly, in various embodiments as otherwise described herein, the catalyst composition includes alkaline earth metal in an amount in the range of 0.1-5 wt %, calculated on an MO basis. For example, in various embodiments, the alkaline earth metal is present in the catalyst in an amount in the range of 0.1-3 wt %, or 0.1-2 wt %, or 0.1-1 wt %, or 0.2-5 wt %, or 0.2-3 wt %, or 0.2-2 wt %, or 0.2-1 wt %, or 0.4-5 wt %, or 0.2-3 wt %, or 0.2-2 wt %, or 0.2-1 wt %, calculated on an MO basis. In some specific embodiments, the alkaline earth metal is present in the catalyst in an amount in the range of 0.2-1.5 wt %, calculated on an MO basis. In some of those embodiments, the alkaline earth metal is present in the catalyst in an amount of 0.2-1 wt %, calculated on an MO basis. Magnesium and calcium are frequent choices of alkaline earth metal additives in the art. Thus in various embodiments the alkaline earth metal is Mg and/or Ca. In some specific embodiments, the alkaline earth metal is Mg. In other specific embodiments, the alkaline earth metal is Ca.

The person of ordinary skill in the art will appreciate that a variety of other components may be present in the catalyst composition. However, the present inventors have noted that advantageous catalyst compositions can be provided substantially from aluminum, zinc, chromium, tin, zirconium, alkali and silicon components (e.g., in the form of oxides). And it is often desirable that light alkane dehydrogenation catalysts be substantially free from components that might impair their yield, selectivity, stability, or other properties. Thus, in various embodiments as otherwise described herein, at least 80 wt %, e.g., at least 85 wt % of the catalyst composition is made up of aluminum, zinc, chromium, tin, zirconium, alkali metal, alkaline earth metal and silicon, each calculated on an oxide basis. In various specific embodiments, at least 90 wt %, e.g., at least 95 wt %, or at least 98 wt %, or at least 99 wt % of the catalyst composition is made up of aluminum, zinc, chromium, tin, zirconium, alkali metal, alkali earth metal and silicon, each calculated on an oxide basis.

Some commercial dehydrogenation catalysts use significant amounts of copper or precious metals such as platinum. However, because of the significant cost of these components, dehydrogenation catalysts that comprise relatively more cost-effective materials are highly desirable. The present inventors have determined that selective dehydrogenation catalysts may be provided without use of substantial amounts of copper. Therefore, in various specific embodiments, the catalyst compositions of the disclosure have no more than 2 wt % copper, e.g., no more than 1 wt %, or no more than 0.5 wt %, or no more than 0.1 wt %, calculated as Cu₂O. The present inventors have similarly determined that selective dehydrogenation catalysts may be provided without use of substantial amounts of platinum, palladium or ruthenium. Thus, in various embodiments the catalyst compositions of the disclosure have no more than 0.1 wt % total of platinum, palladium and ruthenium, e.g., no more than 0.05 wt %, or no more than 0.01 wt %, or no more than 0.005 wt %, or no more than 0.002 wt %, calculated as PtO₂, PdO₂, and RuO₂.

Typical catalyst compositions are based on multicomponent oxides. In various desirable embodiments as otherwise described herein, at least 90 mol % of the metallic species are in oxidic form, e.g., at least 95 mol %, or at least 99 mol %.

The person of ordinary skill in the art can provide catalyst compositions with a variety of surface areas, pore volumes and physical forms, and can select such parameters depending on the particular reaction system in which the catalyst is used. In various embodiments, a catalyst composition of the disclosure has a BET surface area in the range of 60-150 m²/g, In various embodiments, a catalyst composition of the disclosure has a pore volume of 0.2-0.4 mL/g as measured by ASTM D6761-22. The catalyst compositions of the disclosure can be provided in a variety of forms, which will generally depend on the form of the carrier. In various embodiments, the catalyst compositions of the disclosure are provided in the form of pellets or extrudates.

### II. Preparation

The person of ordinary skill in the art can use a variety of methods to prepare the catalyst compositions of the disclosure. For example, in one aspect, the present disclosure provides a method for the preparation of light alkane dehydrogenation catalysts by
providing a calcined zinc-doped alumina carrier; and then
impregnating the zinc-doped alumina carrier with chromium, zirconium, tin, and alkali metal; and then
calcining the impregnated carrier.

The person of ordinary skill in the art can use conventional methods to provide calcined zinc-doped alumina carriers. A number of conventional methods of preparation of alumina from various precursor materials in amounts and concentrations suitable to provide the desired products are known in the art, and the person of ordinary skill in the art, based on the present disclosure, can adapt such methods to provide a zinc-doped alumina carrier.

In various embodiments of this aspect, the zinc-doped alumina material is made by impregnating an alumina material with a zinc source; and calcining the impregnated carrier. The alumina material can be made, e.g., by calcining a mixture of nitric acid and one or more of aluminum trihydroxide and aluminum hydroxide oxide. In other embodiments the preparation of the calcined zinc-doped alumina carrier comprises calcining a mixture of a zinc source, nitric acid and one or more of aluminum trihydroxide and aluminum hydroxide oxide. A variety of zinc sources are known for catalyst synthesis. In various embodiments the zinc source is zinc nitrate or zinc carbonate, but the skilled artisan will appreciate that other compounds of zinc may be used. Calcination during the production of the carrier may be carried out at any temperature between 500° C. and 1300° C., e.g., 500° C. to 1100° C., or 500° C. to 900° C., or 500° C. to 700° C., or 700° C. to 1300° C., or 900° C. to 1300° C., or 1100° C. to 1300° C., or 600° C. to 800° C., or 700° C. to 900° C., or 800° C. to 1000° C., or 900° C. to 1100° C., or 1000° C. to 1200° C., or 1100° C. to 1300° C. The resulting carrier material may then be shaped by any method known in the art, e.g., by extrusion.

Silica can be provided to the zinc-doped alumina carrier using conventional methods, e.g., by including silica or a silica source together with the zinc source, or by using a silica/alumina material in the impregnation.

The calcined zinc-doped alumina carrier can in various embodiments have zinc present predominantly in the form of ZnAl₂O₄, as measured by x-ray photoelectron spectroscopy, as described above for the catalyst compositions of the disclosure. For example, in various embodiments, at least 75 mol % of the zinc of the calcined zinc-doped alumina carrier is present in the form of ZnAl₂O₄, e.g., at least 85 mol % or at least 95 mol %, as measured by x-ray photoelectron spectroscopy.

The zinc-doped alumina carrier is preferably associated with chromium, tin, zirconium, and alkali metal. These components may be introduced to the carrier by, e.g., impregnation, or by any suitable method generally known in the art in one or more steps. Other components, including alkaline earth metal, can similarly be introduced. The components of the catalyst composition are desirably initially provided in their oxidized forms. Oxidation may be achieved by calcining; or by drying and calcining; or by drying, heat treating, and calcining; or by any other suitable method generally known in the art in one or more steps. The final calcination step to produce the catalyst material may be carried out at any temperature between 500° C. and 1300° C., e.g., 500° C. to 1100° C., or 500° C. to 900° C., or 500° C. to 700° C., or 700° C. to 1300° C., or 900° C. to 1300° C., or 1100° C. to 1300° C., or 600° C. to 800° C., or 700° C. to 900° C., or 800° C. to 1000° C., or 900° C. to 1100° C., or 1000° C. to 1200° C., or 1100° C. to 1300° C.

At various points in the preparation of the catalyst, the material may be calcined, as already described. During any of these calcination steps, in certain embodiments the calcination may take place for a period of time in the range of about 10 min. to about 12 hr., or about 15 min. to about 12 hr., or about 20 min. to about 12 hr., or about 30 min. to about 12 hr., or about 45 min. to about 12 hr., or about 1 hr. to about 12 hr., or about 1.5 hr. to about 12 hr., or about 2 hr. to about 12 hr., or about 5 min. to about 11 hr., or about 5 min. to about 10 hr., or about 5 min. to about 9 hr., or about 5 min. to about 8 hr., or about 5 min. to about 7.5 hr., or about 5 min. to about 7 hr., or about 5 min. to about 6.5 hr., or about 5 min. to about 6 hr., or about 5 min. to about 5.5 hr., or about 5 min. to about 5 hr., or about 30 min. to about 11 hr., or about 1 hr. to about 10 hr., or about 1.5 hr. to about 9 hr., or about 2 hr. to about 8 hr.

### III. Use in Dehydrogenation

A major contemplated use of the catalyst compositions provided by the disclosure is to effect the dehydrogenation of light alkanes to the corresponding alkenes. The present inventors have determined that the provided catalyst compositions can advantageously provide improved selectivity over conventional catalysts. Hence, another aspect of the disclosure is a catalytic dehydrogenation process comprising contacting a gaseous aliphatic hydrocarbon stream comprising at least one C₂-C₅ alkane with the catalyst under conditions sufficient to dehydrogenate at least a portion of the C₂-C₅ alkane to one or more corresponding C₂-C₅ alkenes.

In various embodiments as otherwise described herein, the gaseous aliphatic hydrocarbon feed stream comprises one or more C₂-C₅ alkanes. For example, in certain preferred embodiments, at least one C₂-C₅ alkane comprises propane.

The gaseous aliphatic feed stream will often contain air, e.g., provided in a single feed to the reactor or in separate feeds to the reactor. The person of ordinary skill in the art can select an appropriate proportion of air to hydrocarbon in the feed. For example, in some embodiments, the air-to-hydrocarbon ratio of the hydrocarbon feed stream is in the range of 2-6 wt/wt.

The contacting of the feed with the catalyst can be performed using conventional methods. For example, it is typically desirable to use elevated temperatures in the dehydrogenation processes described herein. For example, in various embodiments, the hydrocarbon stream is contacted with the catalyst composition at a temperature in the range of 520-680° C. However, the person of ordinary skill in the art can select other temperatures, depending on a variety of factors. For example, In various embodiments as otherwise described herein, the contacting of the feed is carried out at a temperature within the range of 400° C. to 750° C. For example, in certain embodiments as otherwise described herein, the contacting of the feed with the catalyst is carried out at a temperature within the range of 400° C. to 700° C., or 400° C. to 650° C., or 400° C. to 600° C., or 400° C. to 550° C., or 450° C. to 750° C., or 500° C. to 750° C., or 550° C. to 750° C., or 600° C. to 750° C., or 450° C. to 700° C., or 500° C. to 650° C.

In various embodiments as otherwise described herein, the contacting of the feed is carried out at a pressure within the range of 0.1 bar to 2 bar. For example, in certain embodiments as otherwise described herein, the contacting of the feed is carried out at a pressure within the range of 0.1 bar to 0.9 bar, or 0.1 bar to 0.8 bar, or 0.1 bar to 0.7 bar, or 0.1 bar to 0.6 bar, or 0.1 bar to 0.5 bar, or 0.2 bar to 1 bar, or 0.3 bar to 1 bar, or 0.4 bar to 1 bar, or 0.5 bar to 1 bar, or 0.2 bar to 0.9 bar, or 0.3 bar to 0.8 bar, or 0.4 bar to 0.7 bar, or 0.2 bar to 2 bar, or 0.3 bar to 2 bar, or 0.4 bar to 2 bar, or 0.5 bar to 2 bar, or 0.1 bar to 1.5 bar, or 0.2 bar to 1.5 bar, or 0.3 bar to 1.5 bar, or 0.4 bar to 1.5 bar. In some embodiments, the dehydrogenation is conducted at a pressure in the range of 0.2-2 bar.

The feed may be contacted with the catalyst at a constant rate, or alternatively, at a variable rate. In various embodiments as otherwise described herein, the feed is contacted with the catalyst at a liquid hourly space velocity within the range of 0.5 h⁻¹ to 4 h⁻¹. For example, in certain embodiments as otherwise described herein, the feed is contacted with the catalyst at an space velocity in the range of 0.75 h⁻¹ to 4 h⁻¹, or 1 h⁻¹ to 4 h⁻¹, or 1.25 h⁻¹ to 4 h⁻¹, or 1.5 h⁻¹ to 4 h⁻¹, or 0.5 h⁻¹ to 3.75 h⁻¹, or 0.5 h⁻¹ to 3.5 h⁻¹, or 0.5 h⁻¹ to 3.25 h⁻¹, or 0.5 h⁻¹ to 3 h⁻¹, or 0.5 h⁻¹ to 2.75 h⁻¹, or 0.5 h⁻¹ to 2.5 h⁻¹, or 0.75 h⁻¹ to 3.5 h⁻¹, or 1 h⁻¹ to 3 h⁻¹, or 1.25 h⁻¹ to 2.75 h⁻¹, or 1.5 h⁻¹ to 2.5 h⁻¹. In various embodiments, the feed is contacted with the catalyst at a space velocity in the range of 0.8-2.5 h⁻¹

Dehydrogenation catalysts known in the art are sometimes operated in a mixed-bed system, wherein the first component is a catalytic material and additional components are other catalytic materials or non-catalytic materials. For example, a mixed-bed system might comprise a catalytic material and a heat generating material. Accordingly, in certain embodiments as otherwise described herein, the provided catalyst composition is optionally a component of a mixed-bed system, where another component is a different catalyst composition or a non-catalytic additive. In some specific embodiments, the provided catalyst comprises at least 65% (e.g., at least 75%) by weight of the mixed-bed system.

In general, as the dehydrogenation reaction proceeds, byproducts such as coke will form on the surface of the catalyst, tending to deactivate it over time. If the material has not been otherwise thermally degraded, it can be commercially feasible to regenerate the catalyst and continue using it. This process of deactivation and regeneration may be performed in a plurality of cycles. In some embodiments as otherwise described herein, the catalyst is periodically regenerated by: decoking in the presence of an oxygen-containing gas to remove carbon deposits, e.g. coke, from the catalyst; and contacting the decoked catalyst with hydrogen gas or steam under appropriate conditions generally known in the art. In certain preferred embodiments, a cycle comprising catalytic dehydrogenation and catalyst regeneration is performed a plurality of times.

### EXAMPLES

The following examples are illustrative of specific embodiments of the invention, and various uses thereof. They are set forth for explanatory purposes only, and are not to be taken as limiting the invention. Except as otherwise noted, the person of ordinary skill in the art will appreciate that the syntheses reported employ reagents in amounts and concentrations suitable to provide the desired products.

### Example 1. Preparation and Use of Catalysts Comprising Varying Amounts of Tin and Zinc

A comparative light alkane dehydrogenation catalyst CE1 was made by the following procedure. First, alumina pellets were prepared from a thorough mixing of aluminum trihydroxide and aqueous nitric acid to make wet paste, followed by forming to make pellets of ⅛ inch diameter. The pellets were dried, then heat treated at 450° C. in air, and finally calcined at 616° C. in air. They were then impregnated with a solution of chromic acid, sodium oxide, and zirconium carbonate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, and 0.7 wt % ZrO₂ on alumina.

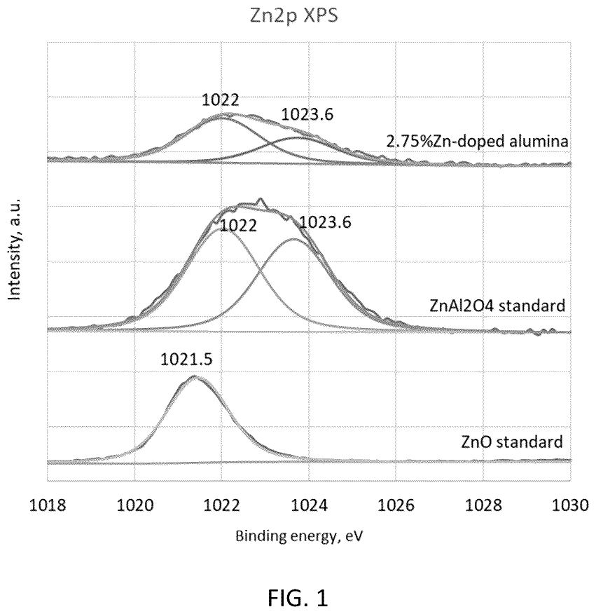

Impregnation of the alumina pellets of CE1 with zinc nitrate after the first calcination step but before impregnation with the solution of chromic acid, sodium oxide, and zirconium carbonate produces Zn-doped alumina pellets. After drying and calcining, the Zn-doped alumina pellets can be analyzed by XPS to determine their composition. A given pure compound containing zinc will produce a characteristic XPS spectrum, corresponding to the chemical state of the zinc, such that an unknown zinc-containing analyte may be compared with known characteristic spectra to determine the form in which zinc is present in the analyte. For example, ZnO produces a single XPS peak centered at 1021.5 eV, while ZnAl₂O₄ produces a curve which can be described by the sum of two Gaussians centered at 1022 eV and 1023.6 eV, respectively. Analyzing the Zn-doped alumina pellets by XPS shows that there is no peak centered at 1021.5 eV, while the curve can be described as the sum of two Gaussians centered at 1022 eV and 1023.6 eV, indicating that zinc is present in the Zn-doped alumina predominantly as ZnAl₂O₄ rather than ZnO. The results of such an analysis are shown in FIG. 1 which provides a comparison of XPS Zn2p spectra between standards and 2.75% Zn-doped alumina (Zn carbonate precursor) after 760° C. calcination.

Light alkane dehydrogenation catalyst A1 was made by first preparing, drying, and calcining alumina pellets by the same method as comparative catalyst CE1. Then, the pellets were impregnated with an aqueous solution of zinc nitrate, followed by air drying at room temperature and calcining at 750° C. for 3 hours, to produce 4 wt % Zn-doped alumina pellets. The Zn-doped alumina pellets were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.64 wt % SnO₂ on 4 wt % Zn-doped alumina.

Light alkane dehydrogenation catalyst A2 was made by first preparing, drying, and calcining alumina pellets by the same method as comparative catalyst CE1. Then, the pellets were impregnated with an aqueous solution of zinc nitrate, followed by air drying at room temperature and calcining at 750° C. for 3 hours, to produce 2 wt % Zn-doped alumina pellets. The Zn-doped alumina pellets were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.32 wt % SnO₂ on 2 wt % Zn-doped alumina.

Light alkane dehydrogenation catalyst A3 was made by first preparing Zn-doped alumina pellets from a thorough mixing of aluminum trihydroxide, zinc nitrate, and aqueous nitric acid to make wet paste. The paste was then formed into pellets of ⅛ in diameter which were dried, then heat treated at 450° C. in air, and finally calcined at 616° C. in air, resulting in 2 wt % Zn-doped alumina pellets. The Zn-doped alumina pellets were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, 0.5 wt % SnO₂ on 2 wt % Zn-doped alumina.

Light alkane dehydrogenation catalyst A4 was made by first preparing Zn-doped alumina pellets from a thorough mixing of aluminum trihydroxide, zinc carbonate, and aqueous nitric acid to make wet paste. The paste was then formed into pellets of ⅛ in diameter which were dried, then heat treated at 450° C. in air, and finally calcined at 616° C. in air, resulting in 1 wt % Zn-doped alumina pellets. The Zn-doped alumina pellets were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, 0.5 wt % SnO₂ on 1 wt % Zn-doped alumina.

Fresh catalysts CE1, A1, A2, A3, and A4 were tested for their propylene selectivity by contacting each with a gaseous feed stream containing propane at a LHSV of 1.5 h⁻¹ at a pressure of 0.5 bar for 300 cycles. The dehydrogenation temperature for each catalyst was varied to achieve a propylene yield close to 39% to be able to make objective propylene selectivity comparisons.

The results of the experiment are shown in Table 1. For each catalyst in the table the alumina precursor was aluminum trihydroxide and each contained 19.6 wt % Cr₂O₃, 0.6 wt % Na₂O, and 0.7 wt % ZrO₂. Compared with conventional catalyst CE1, each of the SnZn-promoted chromia alumina catalysts exhibits improved selectivity toward propylene. The best performer with respect to both yield and selectivity was A4.

TABLE 1
Propylene Selectivity Effects of Variable Zinc and Tin Content

| | | | | | Propylene |
|---|---|---|---|---|---|
| | | | | | selectivity |
| | | | | | improvement |
| | Sn (wt % | Zn (wt % | Zn precursor & | Propylene | relative to |
| Catalyst | as SnO₂) | as ZnO) | doping method | yield, wt % | CE1, wt % |
| CE1 | 0 | 0 | — | 35.4 | — |
| A1 | 0.64 | 4 | Zn(NO₃)₂/impregnation | 37.4 | 2.5 |
| A2 | 0.32 | 2 | Zn(NO₃)₂/impregnation | 36.3 | 3.1 |
| A3 | 0.50 | 2 | Zn(NO₃)₂/blending | 36.3 | 1.4 |
| A4 | 0.50 | 1 | ZnCO₃/blending | 37.4 | 4.4 |

### Example 2. Preparation and Use of Catalysts while Varying Zinc Doping Method

A comparative light alkane dehydrogenation catalyst CE2 was made by the same method as CE1, except that aluminum oxide hydroxide was substituted for aluminum trihydroxide.

Light alkane dehydrogenation catalyst B1 was made by first preparing alumina pellets by the same method as comparative catalyst CE2. Then the pellets were impregnated with an aqueous solution of zinc nitrate, followed by air drying at room temperature and calcining at 750° C. for 3 hours, to produce 2 wt % Zn-doped alumina pellets. They were then further impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.5 wt % SnO₂ on 2 wt % Zn-doped alumina.

Light alkane dehydrogenation catalyst B2 was made by first preparing Zn-doped alumina pellets by a thorough mixing of aluminum oxide hydroxide, zinc nitrate, and aqueous nitric acid to make a wet paste, followed by forming to make pellets of ⅛ inch in diameter; drying said pellets, then heat treating at 450° C. in air, and finally calcining at 616° C. in air to produce 2 wt % Zn-doped alumina pellets. The Zn-doped alumina pellets were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.5 wt % SnO₂ on 2 wt % Zn-doped alumina.

Light alkane dehydrogenation catalyst B3 was made by first preparing Zn-doped alumina pellets by a thorough mixing of aluminum oxide hydroxide, zinc carbonate, and aqueous nitric acid to make a wet paste, followed by forming to make pellets of ⅛ inch in diameter; drying said pellets, then heat treating at 450° C. in air, and finally calcining at 616° C. in air to produce 2 wt % Zn-doped alumina pellets. The Zn-doped alumina pellets were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.5 wt % SnO₂ on 2 wt % Zn-doped alumina.

Fresh catalysts CE2, B1, B2, and B3 were tested for their propylene selectivity by contacting each with a gaseous feed stream containing propane at an LHSV of 1.5 h⁻¹ at a pressure of 0.5 bar for 300 cycles, using similar methods to those described above. The dehydrogenation temperature for each catalyst was varied to achieve a propylene yield close to 39% to be able to make objective propylene selectivity comparisons.

The results of the experiment are shown in Table 2. For each catalyst in the table the alumina precursor was aluminum oxide hydroxide and each contained 19.6 wt % Cr₂O₃, 0.6 wt % Na₂O and 0.7 wt % ZrO₂. Each of the SnZn-promoted catalysts exhibited improved propylene selectivity relative to conventional catalyst CE2. B2 and B3 demonstrated similar selectivity improvements, but B2 in particular also demonstrated a significantly higher yield.

TABLE 2
Effect of Varying Zn Precursor and Doping Method on Propylene Selectivity

| | | | | | Propylene |
|---|---|---|---|---|---|
| | | | | | selectivity |
| | | | | | improvement |
| | Sn (wt % | Zn (wt % | Zn precursor & | Propylene | relative to |
| Catalyst | as SnO₂) | as ZnO) | doping method | yield, wt % | CE1, wt % |
| CE2 | 0 | 0 | — | 33.1 | — |
| B1 | 0.5 | 2 | Zn(NO₃)₂/impregnation | 35.2 | 1.7 |
| B2 | 0.5 | 2 | Zn(NO₃)₂/blending | 37.1 | 6.3 |
| B3 | 0.5 | 2 | ZnCO₃/blending | 33.0 | 5.2 |

### Example 3. Preparation and Use of Catalysts while Varying Zinc Precursor

A comparative light alkane dehydrogenation catalyst CE3 was made by the same method as CE1, except that a mixture of 70% aluminum oxide hydroxide and 30% aluminum trihydroxide was substituted for aluminum trihydroxide.

Light alkane dehydrogenation catalyst C1 was made by first thoroughly mixing 70% aluminum oxide hydroxide with 30% aluminum trihydroxide, zinc nitrate and aqueous nitric acid to make a wet paste, followed by forming to make pellets of ⅛ inch diameter. The pellets were then dried, heat treated at 450° C. in air, and finally calcined at 616° C. in air to make 2 wt % Zn-doped alumina. They were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.5 wt % SnO₂ on 2 wt % Zn-doped alumina.

Light alkane dehydrogenation catalyst C2 was made by first thoroughly mixing 70% aluminum oxide hydroxide with 30% aluminum trihydroxide, zinc carbonate and aqueous nitric acid to make a wet paste, followed by forming to make pellets of ⅛ inch diameter. The pellets were then dried, heat treated at 450° C. in air, and finally calcined at 616° C. in air to make 2 wt % Zn-doped alumina. They were then impregnated with a solution of chromic acid, sodium oxide, zirconium carbonate, and tin oxalate, followed by drying at 120° C. for 10 hours and calcining at 760° C. in 30% steam/70% air atmosphere. The final calcined catalyst had a composition of 19.6 wt % Cr₂O₃, 0.6 wt % Na₂O, 0.7 wt % ZrO₂, and 0.5 wt % SnO₂ on 2 wt % Zn-doped alumina.

Fresh catalysts CE2, C1, and C2 were tested for their propylene selectivity by contacting each with a gaseous feed stream containing propane at an LHSV of 1.5 h⁻¹ at a pressure of 0.5 bar for 300 cycles. The dehydrogenation temperature for each catalyst was varied to achieve a propylene yield close to 39% to be able to make objective propylene selectivity comparisons. The results of the experiment are shown in Table 3. For each catalyst in the table the alumina precursor was 30% aluminum trihydroxide/70% aluminum oxide hydroxide and each contained 19.6 wt % Cr₂O₃, 0.6 wt % Na₂O, and 0.7 wt % ZrO₂.

TABLE 3
Effect of Varying Zn Precursor on Propylene Selectivity.

| | | | | | Propylene |
|---|---|---|---|---|---|
| | | | | | selectivity |
| | | | | | improvement |
| | SnO₂ | ZnO | Zn precursor & | Propylene | relative to |
| Catalyst | (wt %) | (wt %) | doping method | yield, wt % | CE1, wt % |
| CE2 | 0 | 0 | — | 36.5 | — |
| C1 | 0.5 | 2 | Zn(NO₃)₂/ | 35.2 | 1.4 |
| | | | blending | | |
| C2 | 0.5 | 2 | ZnCO₃/ | 37.5 | 4.4 |
| | | | blending | | |

The results demonstrate that both SnZn-promoted catalysts outperform the comparative conventional catalyst CE3 with respect to propylene selectivity. Additionally, choosing zinc carbonate as the zinc precursor relative to zinc nitrate results in improvements to both propylene yield and selectivity.

Various aspects and embodiments are provided by the following enumerated embodiments, which can be combined in any number and in any combination not logically or technically:

Embodiment 1. A dehydrogenation catalyst composition comprising
a zinc-doped alumina carrier; and
associated with the carrier, chromium, tin, zirconium and alkali metal,
wherein the catalyst composition comprises:
   aluminum in an amount in the range of 51-88 wt %, calculated on an Al₂O₃ basis;
   zinc in an amount in the range of 0.1-10 wt %, calculated on a ZnO basis;
   chromium in an amount of 12-30 wt %, calculated on a Cr₂O₃ basis;
   tin in an amount in the range of 0.005-2 wt %, calculated on a SnO₂ basis;
   zirconium in an amount in the range of 0.1-2 wt %, calculated on a ZrO₂ basis; and
   alkali metal in an amount in the range of 0.1-5 wt %, calculated on a M₂O basis.
      Embodiment 2. The catalyst composition of embodiment 1, wherein the aluminum is present in an amount in the range of 51-85 wt %, or 51-82 wt %, or 51-79 wt %, or 60-88 wt %, or 60-85 wt %, or 60-82 wt %, or 60-79 wt %, or 68-88 wt %, or 68-85 wt %, or 68-82 wt %, or 68-79 wt %, or 73-88 wt %, or 73-85 wt %, or 73-82 wt %, or 73-79 wt %, calculated on an Al₂O₃ basis.
      Embodiment 3. The catalyst composition of embodiment 1, wherein the aluminum is present in an amount in the range of 68-85 wt %, calculated on an Al₂O₃ basis.
      Embodiment 4. The catalyst composition of any of embodiments 1-3, wherein zinc is present in an amount in the range of 0.1-8 wt %, or 0.1-6 wt %, or 0.1-4 wt %, calculated on a ZnO basis.
      Embodiment 5. The catalyst composition of any of embodiments 1-3, wherein zinc is present in an amount in the range of 0.5-10 wt %, or 0.5-8 wt %, or 0.5-6 wt %, or 0.5-4 wt %, calculated on a ZnO basis.
      Embodiment 6. The catalyst composition of any of embodiments 1-3, wherein zinc is present in an amount in the range of 1-10 wt %, e.g., 1-8 wt %, or 1-6 wt %, or 1-4 wt %, calculated on a ZnO basis.
      Embodiment 7. The catalyst composition of any of embodiments 1-3, wherein zinc is present in an amount in the range of 2-10 wt %, or 2-8 wt %, or 2-6 wt %, or 2-4 wt %, calculated on a ZnO basis.
      Embodiment 8. The catalyst composition of any of embodiments 1-3, wherein zinc is present in an amount of 0.5-6 wt % (e.g., 1-6 wt %), calculated on a ZnO basis.
      Embodiment 9. The catalyst composition of any of embodiments 1-8, wherein the zinc is present predominantly in the form of ZnAl₂O₄, as measured by x-ray photoelectron spectroscopy.
      Embodiment 10. The catalyst composition of any of embodiments 1-8, wherein at least 75 mol % of the zinc is present in the form of ZnAl₂O₄, e.g., at least 85 mol % or at least 95 mol %, as measured by x-ray photoelectron spectroscopy.
      Embodiment 11. The catalyst composition of any of embodiments 1-10, wherein chromium is present in an amount in the range of 12-28 wt %, or 12-25 wt %, or 12-22 wt %, calculated on a Cr₂O₃ basis.
      Embodiment 12. The catalyst composition of any of embodiments 1-10, wherein chromium is present in an amount in the range of 15-30 wt %, e.g., 15-28 wt %, or 15-25 wt %, or 15-22 wt %, calculated on a Cr₂O₃ basis, for example, 15-25 wt %.
      Embodiment 13. The catalyst composition of any of embodiments 1-10, wherein chromium is present in an amount in the range of 17-30 wt %, e.g., 17-28 wt %, or 17-25 wt %, or 17-22 wt %, calculated on a Cr₂O₃ basis.
      Embodiment 14. The catalyst composition of any of embodiments 1-13, wherein tin is present in an amount in the range of 0.005-1 wt %, e.g., 0.005-0.7 wt %, or 0.005-0.5 wt %.
      Embodiment 15. The catalyst composition of any of embodiments 1-13, wherein tin is present in an amount in the range of 0.05-2 wt %, e.g., 0.05-1 wt, or 0.05-0.7 wt %, or 0.05-0.5 wt %, calculated on a SnO₂ basis.
      Embodiment 16. The catalyst composition of any of embodiments 1-13, wherein tin is present in an amount in the range of 0.1-2 wt %, or 0.1-1 wt %, or 0.1-0.7 wt %, calculated on a SnO₂ basis.
      Embodiment 17. The catalyst composition of any of embodiments 1-13, wherein tin is present in an amount in the range of 0.2-2 wt %, or 0.2-1 wt %, or 0.2-7 wt %, calculated on a SnO₂ basis.
      Embodiment 18. The catalyst composition of any of embodiments 1-13, wherein tin is present in an amount of 0.1-1 wt %, calculated on a SnO₂ basis.
      Embodiment 19. The catalyst composition of any of embodiments 1-18, wherein zirconium is present in an amount in the range of 0.1-1.7 wt %, or 0.1-1.5 wt %, or 0.1-1.2 wt %, or 0.2-2 wt %, or 0.2-1.5 wt %, or 0.2-1.2 wt %, or 0.4-2 wt %, or 0.4-1.5 wt %, or 0.4-1.2 wt %, calculated on a ZrO₂ basis.
      Embodiment 20. The catalyst composition of any of embodiments 1-18, wherein zirconium is present in an amount in the range of 0.2-1.2 wt % on a ZrO₂ basis, calculated on a ZrO₂ basis.
      Embodiment 21. The catalyst composition of any of embodiments 1-20, wherein the alkali metal is present in an amount in the range of 0.1-3 wt %, or 0.1-2 wt %, or 0.1-1 wt %, or 0.2-5 wt %, or 0.2-3 wt %, or 0.2-2 wt %, or 0.2-1 wt %, or 0.4-5 wt %, or 0.2-3 wt %, or 0.2-2 wt %, or 0.2-1 wt %, calculated on an M₂O basis.
      Embodiment 22. The catalyst composition of any of embodiments 1-20, wherein the alkali metal is present in an amount in the range of 0.2-1.5 wt %, calculated on an M₂O basis.
      Embodiment 23. The catalyst composition of any of embodiments 1-20, wherein the alkali metal is present in an amount in the range of 0.2-1 wt %, calculated on an M₂O basis.
      Embodiment 24. The catalyst composition of any of embodiments 1-23, wherein the alkali metal is Na and/or K.
      Embodiment 25. The catalyst composition of any of embodiments 1-23, wherein the alkali metal is Na.
      Embodiment 26. The catalyst composition of any of embodiments 1-23, wherein the alkali metal is K.
      Embodiment 27. The catalyst composition of any of embodiments 1-26, wherein the carrier is a zinc-doped silica/alumina carrier, with silicon present in an amount up to 5 wt %, calculated on an SiO₂ basis.
      Embodiment 28. The catalyst composition of embodiment 27, wherein silicon is present in an amount of up to 3 wt %. e.g., or up to 1 wt %, calculated on an SiO₂ basis.
      Embodiment 29. The catalyst composition of embodiment 27, wherein silicon is present in an amount of 0.1-5 wt %, e.g., 0.1-3 wt %, or 0.1-1 wt %, or 0.2-5 wt %, or 0.2-3 wt %, or 0.2-1 wt %, calculated on an SiO₂ basis.
      Embodiment 30. The catalyst composition of any of embodiments 1-28, having no more than 0.5 wt % silicon, e.g., no more than 0.2 wt %, or no more than 0.1 wt %, or no more than 0.05 wt %, calculated on an SiO₂ basis.
      Embodiment 31. The catalyst composition of any of embodiments 1-30, further comprising alkaline earth metal, present in an amount in the range of 0.1-5 wt %, for example, 0.1-3 wt %, or 0.1-2 wt %, or 0.1-1 wt %, or 0.2-5 wt %, or 0.2-3 wt %, or 0.2-2 wt %, or 0.2-1 wt %, or 0.4-5 wt %, or 0.2-3 wt %, or 0.2-2 wt %, or 0.2-1 wt %, calculated on an MO basis.
      Embodiment 32. The catalyst composition of embodiment 31, wherein the alkaline earth metal is present in an amount in the range of 0.2-1.5 wt %, calculated on an MO basis.
      Embodiment 33. The catalyst composition of embodiment 31, wherein the alkaline earth metal is present in an amount in the range of 0.2-1 wt %, calculated on an MO basis.
      Embodiment 34. The catalyst composition of any of embodiments 31-33, wherein the alkaline earth metal is Mg and/or Ca.
      Embodiment 35. The catalyst composition of any of embodiments 31-33, wherein the alkaline earth metal is Mg.
      Embodiment 36. The catalyst composition of any of embodiments 31-33, wherein the alkaline earth metal is Ca.
      Embodiment 37. The catalyst composition of any of embodiments 1-36, wherein at least 80 wt %, e.g., at least 85 wt % of the catalyst composition is made up of aluminum, zinc, chromium, tin, zirconium, alkali metal, alkaline earth metal and silicon, each calculated on an oxide basis.
      Embodiment 38. The catalyst composition of any of embodiments 1-37, wherein at least 90 wt %, e.g., at least 95 wt %, or at least 98 wt %, or at least 99 wt % of the catalyst composition is made up of aluminum, zinc, chromium, tin, zirconium, alkali metal, alkaline earth metal and silicon, each calculated on an oxide basis.
      Embodiment 39. The catalyst composition of any of embodiments 1-37, having no more than 2 wt % copper, e.g., no more than 1 wt %, or no more than 0.5 wt %, or no more than 0.1 wt %, calculated as Cu₂O.
      Embodiment 40. The catalyst composition of any of embodiments 1-39, having no more than 0.1 wt % total of platinum, palladium and ruthenium, e.g., no more than 0.05 wt %, or no more than 0.01 wt %, or no more than 0.005 wt %, or no more than 0.002 wt %, calculated as PtO₂, PdO₂, and RuO₂.
      Embodiment 41. The catalyst composition of any of embodiments 1-40, wherein at least 90 mol % of the metallic species are in oxidic form, e.g., at least 95 mol %, or at least 99 mol %.
      Embodiment 42. The catalyst composition of any of embodiments 1-41, having a BET surface area in the range of 60-150 m²/g,
      Embodiment 43. The catalyst composition of any of embodiments 1-42, having a pore volume of 0.2-0.4 mL/g as measured by ASTM D6761-22.
      Embodiment 44. The catalyst composition of any of embodiments 1-43, provided in the form of pellets or extrudates
      Embodiment 45. A method for preparing a catalyst composition of any of embodiments 1-55, the method comprising
providing a calcined zinc-doped alumina carrier; and then
impregnating the zinc-doped alumina carrier with chromium, zirconium, tin and alkali metal; then
calcining the impregnated carrier.
   Embodiment 46. The method of embodiment 45, wherein the preparation of the calcined zinc-doped alumina carrier comprises impregnating an alumina material with a zinc source; and calcining the impregnated carrier.
   Embodiment 47. The method of embodiment 46, wherein the alumina material is made by calcining a mixture of nitric acid and one or more of aluminum trihydroxide and aluminum hydroxide oxide.
   Embodiment 48. The method of embodiment 45, wherein the preparation of the calcined zinc-doped alumina carrier comprises calcining a mixture of a zinc source, nitric acid and one or more of aluminum trihydroxide and aluminum hydroxide oxide.
   Embodiment 49. A catalyst composition made by a method of any of embodiments 43-48.
   Embodiment 50. A catalytic dehydrogenation process, the process comprising contacting a gaseous aliphatic hydrocarbon stream comprising at least one C₂-C₅ alkane with a catalyst composition according to any of embodiments 1-44 and 49 under conditions sufficient to dehydrogenate at least a portion of the C₂-C₅ alkane to one or more corresponding C₂-C₅ alkenes.
   Embodiment 51. The process of embodiment 50, wherein the at least one C₂-C₅ alkane comprises propane.
   Embodiment 52. The process of embodiment 50 or embodiment 51, wherein an air-to-hydrocarbon ratio of the hydrocarbon stream is in the range of 2-6 wt/wt.
   Embodiment 53. The process of any of embodiments 50-52, wherein the hydrocarbon stream is contacted with the catalyst composition at a temperature in the range of 520-680° C.
   Embodiment 54. The process of any of embodiments 50-53, wherein the dehydrogenation is conducted at a pressure in the range of 0.2-2.0 bar.
   Embodiment 55. The process of any of embodiments 50-54, wherein the hydrocarbon stream is contacted with the catalyst at an hourly space velocity in the range of 0.8-2.5 h⁻¹.
   Embodiment 56. The process of any of embodiments 50-55, wherein the catalyst is periodically regenerated by
decoking the catalyst in the presence of an oxygen-containing gas to remove coke and/or carbon deposits on the catalyst; and
contacting the decoked catalyst with hydrogen gas or steam.
   Embodiment 57. The process of any of embodiments 50-56, wherein a cycle comprising catalytic dehydrogenation followed by catalyst regeneration is performed a plurality of times.

The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims can, alternatively, be made to depend in the alternative from any preceding claim—whether independent or dependent—and that such new combinations are to be understood as forming a part of the present specification.

1. A dehydrogenation catalyst composition comprising
a zinc-doped alumina carrier;
associated with the carrier, chromium, tin, zirconium and alkali metal,
wherein the catalyst composition comprises:
aluminum in an amount in the range of 51-88 wt %, calculated on an Al₂O₃ basis;
zinc in an amount in the range of 0.1-10 wt %, calculated on a ZnO basis;
chromium in an amount of 12-30 wt %, calculated on a Cr₂O₃ basis;
tin in an amount in the range of 0.005-2 wt %, calculated on a SnO₂ basis;
zirconium in an amount in the range of 0.1-2 wt %, calculated on a ZrO₂ basis; and
alkali metal in an amount in the range of 0.1-5 wt %, calculated on a M₂O basis.

2. The catalyst composition of claim 1, wherein zinc is present in an amount in the range of 0.5-10 wt %, or 0.5-8 wt %, or 0.5-6 wt %, or 0.5-4 wt %, calculated on a ZnO basis.

3. The catalyst composition of claim 1, wherein the zinc is present predominantly in the form of ZnAl₂O₄, as measured by x-ray photoelectron spectroscopy.

4. The catalyst composition of claim 1, wherein at least 75 mol % of the zinc is present in the form of ZnAl₂O₄, e.g., at least 85 mol % or at least 95 mol %, as measured by x-ray photoelectron spectroscopy.

5. The catalyst composition of claim 1, wherein chromium is present in an amount in the range of 15-30 wt %, e.g., 15-28 wt %, or 15-25 wt %, or 15-22 wt %, calculated on a Cr₂O₃ basis, for example, 15-25 wt %.

6. The catalyst composition of claim 1, wherein tin is present in an amount in the range of 0.05-2 wt %, e.g., 0.05-1 wt, or 0.05-0.7 wt %, or 0.05-0.5 wt %, calculated on a SnO₂ basis.

7. The catalyst composition of claim 1, wherein zirconium is present in an amount in the range of 0.2-1.2 wt % on a ZrO₂ basis, calculated on a ZrO₂ basis.

8. The catalyst composition of claim 1, wherein the alkali metal is present in an amount in the range of 0.2-1.5 wt %, calculated on an M₂O basis.

9. The catalyst composition of claim 1, wherein the alkali metal is Na and/or K.

10. The catalyst composition of claim 1, wherein the carrier is a zinc-doped silica/alumina carrier, with silicon present in an amount up to 5 wt %, calculated on an SiO₂ basis.

11. The catalyst composition of claim 1, wherein at least 90 wt %, e.g., at least 95 wt %, or at least 98 wt %, or at least 99 wt % of the catalyst composition is made up of aluminum, zinc, chromium, tin, zirconium, alkali metal, alkaline earth metal and silicon, each calculated on an oxide basis.

12. The catalyst composition of claim 1, further comprising an alkaline earth metal, present in an amount in the range of 0.2-1.5 wt %, calculated on an MO basis.

13. The catalyst composition of claim 12, wherein the alkaline earth metal is Mg and/or Ca.

14. A method for preparing a catalyst composition of claim 1, the method comprising
providing a calcined zinc-doped alumina carrier; and then
impregnating the zinc-doped alumina carrier with chromium, zirconium, tin and alkali metal; then
calcining the impregnated carrier.

15. A catalytic dehydrogenation process, the process comprising contacting a gaseous aliphatic hydrocarbon stream comprising at least one C₂-C₅ alkane with a catalyst composition according to claim 1 under conditions sufficient to dehydrogenate at least a portion of the C₂-C₅ alkane to one or more corresponding C₂-C₅ alkenes.
